(19) United States
(12) Patent Application Publication
(10) Pub. No.: US 2026/0328954 A1
(43) Pub. Date: Oct. 22, 2026

(54) PET APPARATUS WITH HIGH ENERGY RESOLUTION AND FAST TIME RESOLUTION AND DETECTION METHOD THEREOF

(71) Applicant: Zhejiang University of Science and Technology, Hangzhou (CN)

(72) Inventors: Hongsheng SHI, Hangzhou (CN); Jiaxing ZHANG, Hangzhou (CN)

(21) Appl. No.: 19/693,369

(22) Filed: May 30, 2026

(30) Foreign Application Priority Data
Apr. 22, 2026 (CN) 202610537559.0

(51) Int. Cl. G01T 1/29; A61B 6/03

(52) U.S. Cl. CPC G01T 1/2985; A61B 6/037

(57) ABSTRACT

A PET apparatus with high energy resolution and fast time resolution and a detection method thereof, which belongs to the technical field of nuclear medicine detection apparatus. The device includes a detection ring, containing a crystal A with the best energy resolution and a crystal B with the best time resolution, both of which are adjacent. The detection method includes placing the two crystal modules adjacently, scanning the patient in three stages after entering the PET ring: Collecting the signal at rest, rotating the ring to exchange the crystal position to collect the signal, and returning to the original position to collect signals; using the law of drug decay, combined with the energy information of the crystal A and the time information of the crystal B for traceability calculation.

### TECHNICAL FIELD

The present disclosure relates to the technical field of nuclear medicine detection apparatus, in particular to a PET apparatus with high energy resolution and fast time resolution and a detection method thereof.

### BACKGROUND

PET's full name is Positron Emission Tomograph. PET is the most effective medical method for the diagnosis of cancer. It is also highly effective for diagnosing heart disease, pulmonary nodules, thyroid disorders, prostate conditions, Parkinson's disease, rheumatism, depression, Alzheimer's disease, and other common or difficult-to-treat diseases.

In PET diagnosis, patients first take a radioactive drug containing positron-emitting nuclides (such as fluorine-18, carbon-11, oxygen-15, nitrogen-13, etc.). This drug follows a strict decay law to emit positrons. Each positron quickly annihilates with an electron in human tissue, emitting two gamma photons with an energy of 511 keV in opposite directions (approximately 1800 apart). The detection ring of the PET scanner can receive these two oppositely directed gamma photons. This detection ring contains a nuclear radiation detection crystal, which reads the total energy of the gamma photons. Electrons in lesions such as tumors are more active. Therefore, by tracing the collected data signals and establishing algorithms, tissue imaging with significant brightness differences can be formed to diagnose the lesion site. In this process, the better the energy resolution of the nuclear radiation detection crystal, the more sensitive the detector and the more accurate the diagnosis.

TOF-PET is an upgraded version of PET. Its full name is Time of Flight-Positron Emission Tomograph. Because the arrival times of the two oppositely directed gamma photons emitted from the lesion to the detection ring may differ, the time resolution of the crystal can be used to further localize the lesion area, obtaining clearer medical images. TOF-PET is currently the mainstream product of PET. The time resolution of TOF-PET is referred to as coincidence time resolution (CTR). In this process, the better the coincidence time resolution of the nuclear radiation detection crystal, the more accurate the detector's diagnosis.

At present, the crystal with the best energy resolution for PET at room temperature is the CZT (cadmium zinc telluride) crystal, but the coincidence time resolution of the CZT crystal is very poor. LYSO and LaBr₃ crystals have the best time resolution for TOF-PET, but their energy resolution is not as good as that of CZT crystals. This situation is not accidental.

The CZT crystal is a semiconductor crystal. When a gamma photon strikes the crystal, electron-hole pairs are generated. The device at the back end of the crystal collects the current signal generated during the recombination of these electron-hole pairs, and the total energy of the gamma photon can be calculated from this signal. This detection method is known as direct detection. LYSO and LaBr₃ crystals are inorganic scintillator crystals. When a gamma photon strikes these crystals, visible light is generated. A photomultiplier tube, silicon photomultiplier tube, or avalanche photodiode at the back end of the crystal converts the visible light into an electrical signal, from which the total energy of the gamma photon is then calculated. This detection method is known as indirect detection. The bandgap of semiconductor crystals is significantly smaller than that of inorganic scintillator crystals, and the energy required to generate each electron-hole pair is also significantly smaller. This means that for the same total energy, the current signal generated is stronger. Therefore, the crystals with the best energy resolution are semiconductor crystals, such as CZT and high-purity germanium (Ge) crystals. However, the coincidence time resolution of semiconductor crystals depends on the electrical signal, which is relatively slow. Inorganic scintillator crystals require more energy to generate each electron-hole pair, so their energy resolution is not as good as that of semiconductor crystals. In PET, for inorganic scintillator crystals, the time resolution depends on the optical signal, making it significantly faster.

### SUMMARY

The purpose of the present disclosure is to provide a PET apparatus with high energy resolution and fast time resolution and a detection method thereof, and to solve the technical problem that the same detection material cannot obtain the best energy resolution and the fastest time resolution at the same time due to the inherent properties of the material in the existing technology.

In order to achieve the above purpose, the present disclosure provides a PET device with high energy resolution and fast time resolution, including a detection ring, the detection ring contains two crystals; one crystal is crystal A with a best energy resolution, and the other crystal is crystal B with a best time resolution, the two crystals are adjacently distributed in a PET machine.

In the PET detection and diagnosis, according to the size of the crystal module, the large ring containing the crystal first collects the signal of the first time interval t₁ in a fixed position, and then the large ring containing the crystal rotates at a specific angle, so that the crystal A is in the original position of the crystal B, and then collects the signal of the second time interval t₂, and then returns to the original position to collect the signal of the third time interval t₃. Since the positron emitted by the drug taken by the patient is emitted from a specific nuclide, its attenuation law conforms to the strict mathematical law. Therefore, the energy signal of the required time interval can be simulated from the time intervals t₁, t₂, or t₃, and then the energy signal is combined with the time signal to obtain the best energy resolution and the best time resolution at the same position, and the clearest image of the lesion can be obtained.

In some embodiments, crystal A is a semiconductor crystal for collecting energy signals; crystal B is an inorganic ion crystal for collecting time signals.

In some embodiments, the crystal A is a cadmium zinc telluride CZT crystal, and the crystal B is yttrium lutetium silicate LYSO:Ce crystal or lanthanum bromide LaBr₃:Ce crystal.

In some embodiments, the PET device combines the energy information of crystal A with the best energy resolution and time information of crystal B with the best time resolution to perform traceability calculation and obtain diagnostic information of a tumor.

In some embodiments, a detection module containing crystal A and a detection module containing crystal B are placed adjacent to each other and made into a detector, where the cross-sectional dimensions of pixel units of crystal A and crystal B are the same.

In some embodiments, the cross-sectional dimensions of the single detection module containing crystal A and the detection module containing crystal B are 11 mm×11 mm or 16 mm×16 mm; each module is divided into 16 pixels, and the cross-sectional dimension of a single pixel is 2.75 mm×2.75 mm or 4 mm×4 mm.

In some embodiments, when the detection module uses a CZT crystal, a full-scale depth of a single pixel is 4 mm; when the detection module uses LYSO:Ce crystal, a full-size depth of a single pixel is 3 mm; when the detection module uses LaBr₃:Ce crystal, a full-size depth of a single pixel is 4 mm.

The present disclosure also provides a detection method for the PET apparatus with high energy resolution and fast time resolution, including the following steps:
S1, making a detection module by using crystal A with the best energy resolution;
S2, making a detection module by using crystal B with the best time resolution; the cross-sectional dimensions of the pixel units of crystal A and crystal B in the detection module are the same;
S3, placing the detection module containing crystal A and the detection module containing crystal B adjacent to each other and fabricating them into a detector;
S4, after a patient took the medicine and PET is operated, the apparatus first remains stationary while scanning the patient during the first time interval t₁, and then rotating the detector to switch positions of adjacent detection modules composed of the two crystals, and then scanning the patient for the second time interval t₂, and then rotating the detector in an opposite direction to make it return to an original position, and then scanning the patient for the third time interval t₃;
S5, according to a positron decay law of nuclides taken by patients and the energy signal detected by the detection module containing crystal A in a specific time interval, calculating the energy signal in a whole time interval, obtaining the energy signal of crystal B with the best time resolution in the detection period, and drawing a diagnostic image by combining the best energy resolution and the best time resolution.

In some embodiments, in step S4, time lengths of the first time interval t₁, the second time interval t₂, and the third time interval t₃ are the same or different, and a size of the specific rotation angle depends on a diameter of the detection ring and a size of the crystal module.

Therefore, the present disclosure adopts a PET apparatus with high energy resolution and fast time resolution and a detection method thereof of the above structure, which has the following beneficial effects:
(1) The present disclosure solves the technical problem that, due to the inherent properties of materials in existing technologies, the same detection material cannot achieve both optimal energy resolution and optimal time resolution simultaneously. By employing a composite detection structure comprising crystal A and crystal B, the PET device can possess both the best energy resolution and the best time resolution.
(2) The present disclosure achieves effective fusion and traceability calculation of the high-precision energy signals collected by crystal A and the high-precision time signals collected by crystal B, by utilizing the specific rotation mode of the detection ring and the decay of the positron radiation source in accordance with mathematical laws. This ensures the accuracy of diagnostic data.
(3) The present disclosure can obtain the best energy resolution and the best time resolution at the same spatial position, thereby producing the clearest image of the lesion. This significantly improves the detection accuracy of PET for tumors and other diseases and offers better clinical application value.

The following is a further detailed description of the technical scheme of the present disclosure through drawings and implementation examples.

### BRIEF DESCRIPTION OF THE DRAWINGS

FIG. 1 is a section diagram of the detection module used in the present disclosure;

FIG. 2 is a cross-section diagram of a single detection module containing crystal A used in the present disclosure;

FIG. 3 is a frame diagram of a single detection module containing a semiconductor crystal used in the present disclosure;

FIG. 4 is a cross-section diagram of a single detection module containing crystal B used in the present disclosure;

FIG. 5 is a frame diagram of a single detection module containing inorganic ion crystals used in the present disclosure.

### DETAILED DESCRIPTION

The following is a further explanation of the technical scheme of the present disclosure through drawings and implementation examples.

Unless otherwise defined, the technical terms or scientific terms used in the present disclosure should be understood by people with general skills in the field to which the present disclosure belongs. The words ‘first’, ‘second’, and the like used in this present disclosure do not represent any order, quantity, or importance, but are only used to distinguish different components. Similar words, such as ‘include’ or ‘comprise’, mean that the elements or objects appearing before the word cover the elements or objects listed after the word and their equivalents, without excluding other elements or objects. Similar words such as ‘connected’ or ‘connecting’ are not limited to physical or mechanical connections, but can include electrical connections, whether direct or indirect. ‘Up’, ‘down’, ‘left’, ‘right’, etc. are only used to represent the relative positional relationship. When the absolute position of the described object changes, the relative positional relationship may also change accordingly.

The present disclosure provides a PET apparatus with high energy resolution and fast time resolution, there are two kinds of detection crystals in the device, one is crystal A with the best energy resolution, and the other is crystal B with the best time resolution, the detection modules composed of these two crystals are placed adjacent.

After the patient takes the medicine and enters the PET detector, the PET first stays still and scans the patient for a short period of time t₁, and then rotates the detector to make the adjacent detection modules composed of the two crystals exchange positions, and then scans the patient for a short period of time t₂, and then reversely rotates the detector at the same angle to return it to the original position, and then scans the patient for a short period of time t₃. Since the positron emitted by the drug taken by the patient is emitted from a specific nuclide, its attenuation law conforms to the strict mathematical law. Therefore, the energy signal of the required time interval can be simulated from the t₁, t₂, or t₃ time interval, and then the energy signal is combined with the time signal to obtain the best energy resolution and the best time resolution at the same position, and the clearest image of the lesion can be obtained.

The present disclosure also provides a detection method of the PET apparatus with both high energy resolution and fast time resolution. The specific operation is carried out according to the following steps:
S1, making a detection module by using crystal A with the best energy resolution;
S2, making a detection module by using crystal B with the best time resolution; the cross-sectional dimensions of the pixel units of crystal A and crystal B in the detection module are the same;
S3, placing the detection module containing crystal A and the detection module containing crystal B adjacent to each other and fabricating them into a detector;
S4, after a patient took the medicine and PET is operated, the apparatus first remains stationary while scanning the patient during the first time interval t₁, and then rotating the detector to switch positions of adjacent detection modules composed of the two crystals, and then scanning the patient for the second time interval t₂, and then rotating the detector in an opposite direction to make it return to an original position, and then scanning the patient for the third time interval t₃;
S5, according to a positron decay law of nuclides taken by patients and the energy signal detected by the detection module containing crystal A in a specific time interval, calculating the energy signal in a whole time interval, obtaining the energy signal of crystal B with the best time resolution in the detection period, and drawing a diagnostic image by combining the best energy resolution and the best time resolution.

### Embodiment 1

The construction, diagnosis, and treatment method of a certain type of PET:

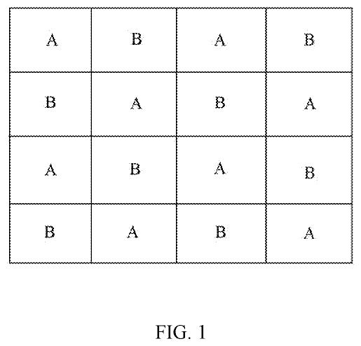

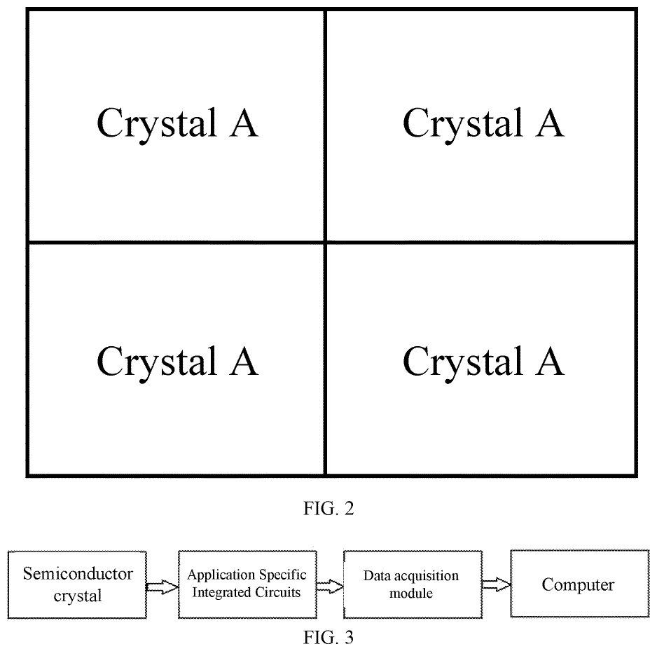

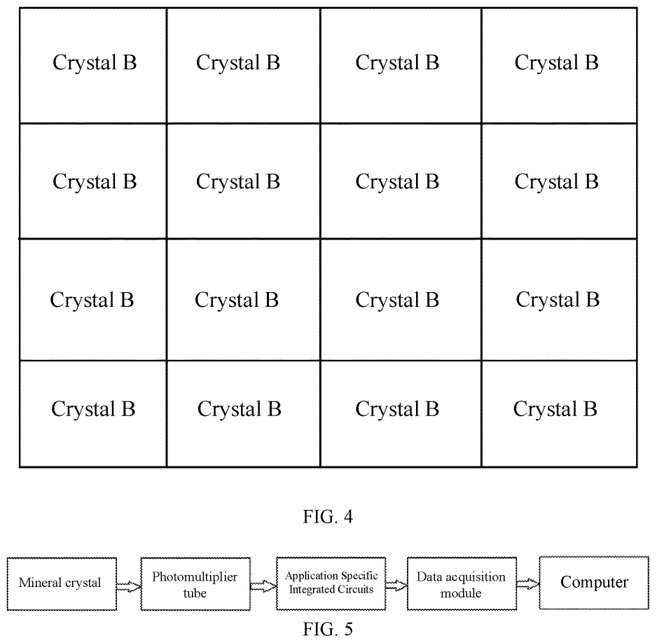

The constructed PET contains a CZT detection module and a LYSO:Ce detection module. The cross-sectional dimension of a single detection module is 11 mm×11 mm, each module is divided into 16 pixels, and the cross-sectional dimension of a single pixel is 2.75 mm×2.75 mm, the full size of a single pixel in the CZT detection module is 2.75 mm×2.75 mm×4 mm, and the full size of a single pixel in the LYSO:Ce detection module is 2.75 mm×2.75 mm×3 mm. The two detection modules are adjacent to each other, as shown in FIG. 1; wherein the CZT detection module adopts a single detection module structure containing semiconductor crystals, as shown in FIG. 2 and FIG. 3; the LYSO:Ce detection module adopts a single detection module structure containing inorganic ion crystals as shown in FIG. 4 and FIG. 5.

The diameter of the PET is 70 cm, and the distance between the center of the PET detection ring and the center of the surface of a single detection module is 36 cm.

The drug taken by the patient is ¹⁸F-FDG, after the patient takes the drug and enters the PET ring, PET first scans the patient for 4 minutes t₁, then rotates clockwise 1° 45′, and then scans the patient for 4 minutes t₂, then rotates counterclockwise 1° 45′, and then scans the patient for 4 minutes t₃, and then processes the data.

The nuclide that releases positrons in the drug ¹⁸F-FDG is ¹⁸F, the decay reaction equation of ¹⁸F is as follows:

$${}_{9}^{18} F \to {}_{8}^{18} O + {e}^{+} + {v}_{e} ; \qquad ( 1 )$$

the half-life of ¹⁸F decay in Equation (1) is 109.7 minutes, according to this decay law and the energy detected by the CZT detection module in a specific time interval, the energy of the detection module in all time intervals is calculated to obtain the best energy resolution. Combined with the time resolution of the LYSO:Ce detection module, and corresponding to the specific part of the detection cross section, the best energy resolution and the best time resolution can be combined to obtain the image.

### Embodiment 2

The construction, diagnosis, and treatment methods of a certain type of PET:

The constructed PET contains a CZT detection module and a LYSO:Ce detection module. The cross-sectional dimension of a single detection module is 16 mm×16 mm. Each module is divided into 16 pixels, and the cross-sectional dimension of a single pixel is 4 mm×4 mm. The full size of a single pixel in the CZT detection module is 4 mm×4 mm×4 mm, and the full size of a single pixel in the LYSO:Ce detection module is 4 mm×4 mm×3 mm; the two detection modules are adjacent to each other as shown in FIG. 1, among them, the CZT detection module adopts a single detection module structure containing semiconductor crystals as shown in FIG. 2 and FIG. 3; the LYSO:Ce detection module adopts a single detection module structure containing inorganic ion crystals as shown in FIG. 4 and FIG. 5.

The diameter of the PET is 70 cm. The distance between the center of the PET detection ring and the center of the surface of a single detection module is 36 cm. The detection ring contains a small baffle that can be automatically controlled, which can determine the position of the ring.

The medicine patient took is ¹⁸F-FDG. After the patient took the medicine and entered the PET ring, PET first scans the patient for 5 minutes t₁, then removes the baffle, rotates about 2° 32′ 45″ clockwise, and then fixes the baffle to ensure that the CZT crystal and LYSO:Ce crystal reaches a strict position exchange, and then scans the patient for 5 minutes t₂, then removes the baffle, rotates about 2° 32′ 45″ counterclockwise, and then fixed the baffle to ensure that the CZT crystal and LYSO:Ce crystal returns to the initial position, and then scans the patient for 5 minutes t₃, and then processes the data, the processing method is the same as that of embodiment 1, and the image is obtained.

### Embodiment 3

The construction, diagnosis, and treatment method of a certain type of PET:

The constructed PET contains a CZT detection module and a LYSO:Ce detection module, the cross-sectional dimension of a single detection module is 16 mm×16 mm, each module is divided into 16 pixels, and the cross-sectional dimension of a single pixel is 4 mm×4 mm; the full size of a single pixel in the CZT detection module is 4 mm×4 mm×4 mm, and the full size of a single pixel in the LYSO:Ce detection module is 4 mm×4 mm×3 mm. The two detection modules are adjacent to each other, as shown in FIG. 1. Among them, the CZT detection module adopts a single detection module structure containing semiconductor crystals, as shown in FIG. 2 and FIG. 3; the LYSO:Ce detection module adopts a single detection module structure containing inorganic ion crystals as shown in FIG. 4 and FIG. 5.

The diameter of the PET is 80 cm, and the distance between the center of the PET detection ring and the center of the surface of a single detection module is 41 cm.

The patient is treated with ¹⁸F-FDG, after the patient takes the drug and enters the PET ring, the PET first scans the patient for 5 minutes t₁, then rotates 2° 17′29″ clockwise, and then scans the patient for 5 minutes t₂, then rotates 2° 17′29″ counterclockwise, and then scans the patient for 5 minutes t₃, and then processes the data. The processing method is the same as Embodiment 1, and the image is obtained.

### Embodiment 4

The construction, diagnosis, and treatment method of a certain type of PET:

The constructed PET contains a CZT detection module and a LaBr₃:Ce detection module. The cross-sectional dimension of a single detection module is 16 mm×16 mm. Each module is divided into 16 pixels, and the cross-sectional dimension of a single pixel is 4 mm×4 mm, the full size of a single pixel in the CZT and LaBr₃:Ce detection modules is 4 mm×4 mm×4 mm, the two detection modules are adjacent to each other as shown in FIG. 1. Among them, the CZT detection module adopts a single detection module structure containing semiconductor crystals as shown in FIG. 2 and FIG. 3; the LaBr₃:Ce detection module adopts a single detection module structure containing inorganic ion crystals as shown in FIG. 4 and FIG. 5.

The diameter of the PET is 70 cm, the distance between the center of the PET detection ring and the center of the surface of a single detection module is 36 cm, and the detection ring contains a device that can accurately fix the position of the ring.

The patient's drug is ¹⁸F-FDG. After the patient took the drug and entered the PET ring, the PET first scans the patient for 5 minutes t₁, and then rotates the ring clockwise to make the CZT crystal and LaBr₃:Ce crystal achieve strict position exchange, and then scans the patient for 5 minutes t₂, and then rotates the ring counterclockwise to ensure that the CZT crystal and LaBr₃:Ce crystal returned to the initial position, and then scans the patient for 5 minutes t₃, and then processes the data, the processing method is the same as Embodiment 1, and the image is obtained.

### Embodiment 5

The construction, diagnosis, and treatment method of a certain type of PET:

The constructed PET contains a CZT detection module and a LaBr₃:Ce detection module, the cross-sectional dimension of a single detection module is 16 mm×16 mm, each module is divided into 16 pixels, and the cross-sectional dimension of a single pixel is 4 mm×4 mm, the full size of a single pixel in the CZT and LaBr₃:Ce detection modules is 4 mm×4 mm×4 mm, the two detection modules are adjacent to each other as shown in FIG. 1. Among them, the CZT detection module adopts a single detection module structure containing semiconductor crystals as shown in FIG. 2 and FIG. 3; the LYSO:Ce detection module adopts a single detection module structure containing inorganic ion crystals as shown in FIG. 4 and FIG. 5.

The diameter of the PET is 80 cm, and the distance between the center of the PET detection ring and the center of the surface of a single detection module is 41 cm.

The patient is treated with ¹⁸F-FDG. After the patient took the drug into the PET ring, the PET first scans the patient for 5 minutes t₁, then rotates 2° 17′29″ clockwise, and then scans the patient for 5 minutes t₂, then rotates 2° 17′29″ counterclockwise, and then scans the patient for 5 minutes t₃, and then processes the data, the processing method is the same as Embodiment 1, and the image was obtained.

Finally, it should be explained that the above embodiments are only used to explain the technical scheme of the present disclosure rather than restrict it. Although the present disclosure is described in detail with reference to the better embodiment, the ordinary technical personnel in this field should understand that they can still modify or replace the technical scheme of the present disclosure, and these modifications or equivalent substitutions cannot make the modified technical scheme out of the spirit and scope of the technical scheme of the present disclosure.

What is claimed is:

1. A PET device with high energy resolution and fast time resolution, comprising a detection ring, wherein the detection ring comprises two crystals; one crystal is crystal A with a best energy resolution, and the other crystal is crystal B with a best time resolution, the two crystals are adjacently distributed in a PET machine;
in a PET detection and diagnosis, according to a size of the crystal module, the large ring containing the crystal first collects a signal of a first time interval in a fixed position, and then the large ring containing the crystal rotates at a specific angle, so that the crystal A is in an original position of the crystal B, and then collects a signal of a second time interval, and then returns to the original position to collect a signal of a third time interval.

2. The PET device with high energy resolution and fast time resolution according to claim 1, wherein the crystal A is a semiconductor crystal for collecting energy signals; the crystal B is an inorganic ion crystal for collecting time signals.

3. The PET device with high energy resolution and fast time resolution according to claim 1, wherein the crystal A is a cadmium zinc telluride CZT crystal, and crystal B is yttrium lutetium silicate LYSO:Ce crystal or lanthanum bromide LaBr₃:Ce crystal.

4. The PET device with high energy resolution and fast time resolution according to claim 1, wherein the PET device combines energy information of crystal A with the best energy resolution and time information of crystal B with the best time resolution to perform traceability calculation and obtain diagnostic information of a tumor.

5. The PET device with high energy resolution and fast time resolution according to claim 1, wherein a detection module containing the crystal A and a detection module containing the crystal B are placed adjacent and made into a detector, where the cross-sectional dimensions of pixel units of crystal A and crystal B are the same.

6. The PET device with high energy resolution and fast time resolution according to claim 5, wherein the cross-sectional dimensions of the single detection module containing the crystal A and the detection module containing the crystal B are 11 mm×11 mm or 16 mm×16 mm; each module is divided into 16 pixels, and a cross-sectional dimension of a single pixel is 2.75 mm×2.75 mm or 4 mm×4 mm.

7. The PET device with high energy resolution and fast time resolution according to claim 6, wherein when the detection module uses CZT crystal, a full-scale depth of a single pixel is 4 mm; when the detection module uses LYSO:Ce crystal, a full-size depth of a single pixel is 3 mm; when the detection module uses LaBr₃:Ce crystal, a full-size depth of a single pixel is 4 mm.

8. A detection method for the PET apparatus with high energy resolution and fast time resolution according to claim 1, comprising the following steps:
S1, making a detection module by using the crystal A with the best energy resolution;
S2, making a detection module by using the crystal B with the best time resolution; the cross-sectional dimensions of the pixel units of crystal A and crystal B in the detection module are the same;
S3, placing the detection module containing the crystal A and the detection module containing the crystal B adjacent to each other and fabricating them into a detector;
S4, after a patient took the medicine and PET is operated, the apparatus first remains stationary while scanning the patient during a first time interval t₁, and then rotating the detector to switch positions of adjacent detection modules composed of the two crystals, and then scanning the patient for a second time interval t₂, and then rotating the detector in an opposite direction to make it return to an original position, and then scanning the patient for a third time interval t₃;
S5, according to a positron decay law of nuclides taken by patients and the energy signal detected by the detection module containing the crystal A in a specific time interval, calculating an energy signal in a whole time interval, obtaining the energy signal of the crystal B with the best time resolution in the detection period, and drawing a diagnostic image by combining the best energy resolution and the best time resolution.

9. The PET device with high energy resolution and fast time resolution according to claim 8, wherein in step S4, time lengths of the first time interval t₁, the second time interval t₂, and the third time interval t₃ are the same or different, and a size of a specific rotation angle depends on a diameter of the detection ring and a size of the crystal module.
